(19) United States
(12) Patent Application Publication
(10) Pub. No.: US 2004/0044392 A1
(43) Pub. Date: Mar. 4, 2004

(54) Stent catheter system

(75) Inventor: Randolf Von Oepen, Hirrlingen (DE)

Correspondence Address: SHINJYU GLOBAL IP COUNSELORS, LLP, 1233 20TH STREET, NW, SUITE 700, WASHINGTON, DC, 20036-2680, US

(73) Assignee: Jomed GmbH, Rangendingen (DE)

(21) Appl. No.: 10/652,498

(22) Filed: Sep. 2, 2003

Related U.S. Application Data
(63) Continuation of application No. 09/720,873, filed on Mar. 16, 2001.
Application No. 09/720,873 is a 371 of international application No. PCT/EP00/03971, filed on May 3, 2000.

(30) Foreign Application Priority Data
May 3, 1999 (DE) 299 07 827.2

(51) Int. Cl. A61F 2/06

(52) U.S. Cl. 623/1.1

(57) ABSTRACT

To effect a throttling of the blood flow which with some indications is required in vessels, a stent-catheter arrangement (1) according to the invention comprises a catheter with an expandable balloon (3) and a stent (4) which can be placed on the balloon. The stent (4) is provided with a cover and the balloon (3) has a section (5) of reduced expandability approximately in the central portion thereof. When the balloon is expanded, it is thus possible to form a throttle section of the stent which is created at the place where the balloon (3) includes a section (5) of reduced expandability.

### DESCRIPTION

The present invention relates to a stent-catheter arrangement according to the preamble of claim 1.

A generic arrangement is for example used for expanding stenoses in blood vessels. To this end the stent, which is arranged on the expandable balloon of the stent-catheter arrangement, is introduced into the blood vessel of a patient and pushed forwards into the area of stenosis with the help of a guide wire. After the stent has been placed inside the stenosis, the balloon is expanded by filling a suitable medium into the catheter, whereby the stent will also extend radially until the stenosis is expanded to the desired degree. The balloon is then emptied and the catheter arrangement can be withdrawn from the patient's body, with the stent remaining within the stenosis, thereby permanently keeping the same in the expanded position.

Studies carried out within the scope of the invention have however shown that such an arrangement cannot be used with indications in which the blood flow must deliberately be throttled in the blood vessel of a patient, normally in newborns, babies, children and infants, because the generic stent-catheter arrangement is only suited for effecting the opposite of such a throttling action, i.e. the expansion of a vessel and thus the elimination of a constriction.

It is therefore the object of the invention to provide a stent-catheter arrangement of the type indicated in the preamble of claim 1, with which it is possible to throttle the blood flow in blood vessels.

This object is achieved by the features of claim 1.

The stent-catheter arrangement according to the invention comprises a catheter which includes an expandable balloon which on at least one of its portions, normally approximately in its central portion, has a section of reduced expandability. The balloon designed in this way has normally mounted, e.g. crimped, thereon in the conventional way a stent which is provided with a cover that is impermeable to flow.

When this arrangement is introduced into a patient's vessel and when the balloon is expanded, one will obtain a stent structure which remains in the vessel and comprises two portions by means of which the stent is fixed within the vessel. When the balloon is expanded, a throttle section is obtained between said two fixing portions because in this area the stent rests on the balloon portion of reduced expandability. Depending on the respective treatment, the throttle portion may be designed differently but at any rate has a volume which permits a blood flow which, though it is throttled, is still adequate.

Preferably, the fixing portions and the throttle portion of the stent have disposed thereinbetween a gradually tapering portion, so that a whirling of the blood flow can be avoided at least to a substantial degree.

The subclaims refer to advantageous developments of the invention.

The cover of the stent can be obtained by means of a suitable body-tolerated foil which may e.g. consist of PTFE material.

The balloon portion of reduced expandability can be obtained through a suitable stiffening of the balloon material in said section; to this end use can e.g. be made in the balloon material of integrated stiffenings or also of stiffenings applied to the balloon material in said portion, e.g. a stiffening ring preferably adhesively attached thereto.

Furthermore, it is possible to form the balloon portion of reduced expandability during balloon production. To this end the metal form which is provided for producing the balloon and into which the balloon material is blown may be equipped with a cross-sectional constriction at the place where the balloon portion of reduced expandability is to be arranged at a later time.

Further details, features and advantages of the invention will become apparent from the following description of an embodiment taken in conjunction with the drawing, in which:

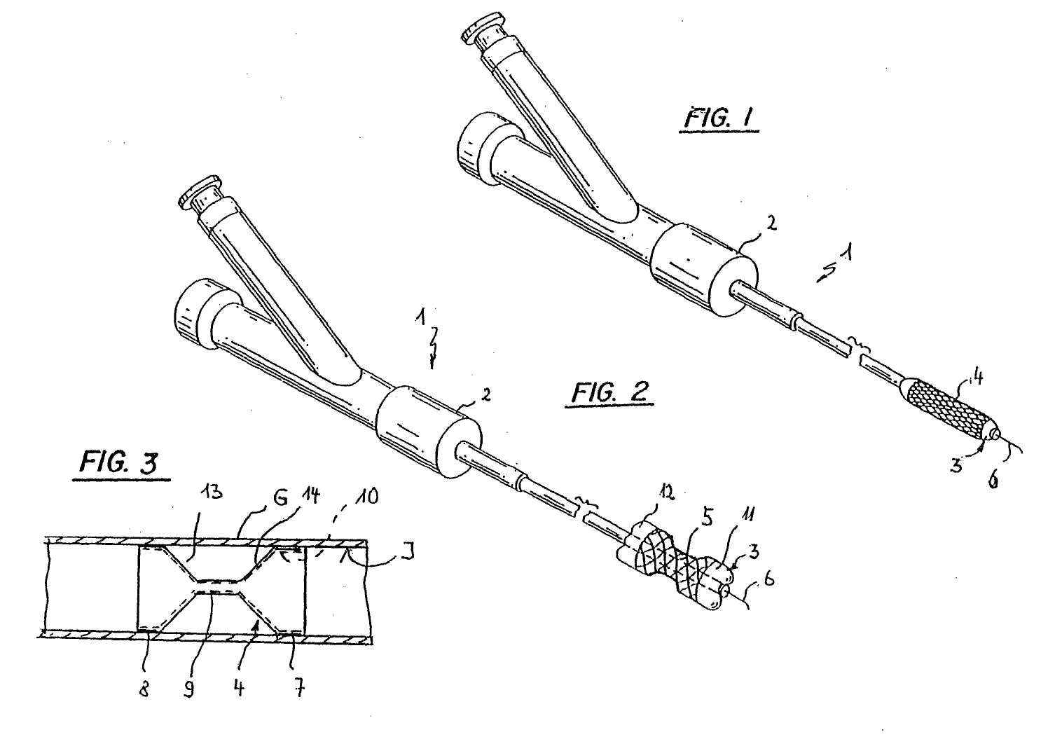

FIG. 1 is a perspective view of a stent-catheter arrangement according to the invention, with a stent being placed on a balloon in the non-expanded state;

FIG. 2 is a view of the inventive stent-catheter arrangement corresponding to FIG. 1, in which both the balloon and the stent are shown in the expanded state, and

FIG. 3 is a schematically strongly simplified view showing a blood vessel with the stent of the invention arranged therein.

FIGS. 1 and 2 show a possible embodiment of a stent-catheter arrangement 1 according to the invention. The arrangement 1 may include a commercially designed catheter 2 with an expandable balloon 3. In the illustration of FIG. 1 a stent 4 is placed, e.g. crimped, on the balloon 3 in the non-expanded state.

The balloon 3 of the stent-catheter arrangement according to the invention includes a section 5 of reduced expandability which is visible in FIG. 2 in which the total expanded state of the balloon 3 is shown. The section 5 of reduced expandability is positioned between two fully expandable sections 11 and 12.

Apart from further features of the catheter 2, which are known per se, FIGS. 1 and 2 show a guide wire 6 with the help of which the stent 4 can be placed in the vessel of a patient.

FIG. 3 shows this state. FIG. 3 is a schematically simplified view of a patient's vessel G in which a stent 4 according to the invention is arranged. With the expansion of the balloon 3, two fixing sections 7 and 8 have been obtained by which the stent 4 is fixed in vessel G, i.e. on the inner wall I thereof. A throttle portion 9 of a clearly reduced cross-section in comparison with the fixing sections 7 and 8 is positioned between the fixing portions 7 and 8. Such a reduction is created by the less expandable section 5 of the balloon 3 upon expansion of the balloon 3, as illustrated in FIG. 2.

A tapering section 13 and 14 is respectively positioned between the fixing sections 7 and 8 and the throttle section 9.

Furthermore, FIG. 3 is a simplified view showing a cover (coating) 10 which covers the whole stent structure, which is normally a web structure, so that the web structure of the stent 4 of the invention which is per se permeable to blood is made impermeable to liquids.

1. A stent-catheter arrangement (1) comprising
a catheter (2) including an expandable balloon (3);
a stent (4) which can be placed on said expandable balloon (3), characterized in
that said stent (4) is provided with a cover (10), and
that said balloon (3) has provided between the ends thereof a section (5) of reduced expandability in comparison with fully expandable sections (11, 12).

2. The stent-catheter arrangement according to claim 1, characterized in that said cover (10) is a foil or a coating.

3. The stent-catheter arrangement according to claim 2, characterized in that said foil or said coating is made from body-tolerated material.

4. The stent-catheter arrangement according to claims 2 or 3, characterized in that said foil or said coating consists of biological material, of polymer material, of metallic material, ceramic material or elastomer material.

5. The stent-catheter arrangement according to any one of claims 1 to 3, characterized in that said section (5) of said balloon (3) which is expandable to a reduced degree consists of stiffened balloon material.

6. The stent-catheter arrangement according to any one of claims 1 to 3, characterized in that said section (5) of said balloon (3) which is expandable to a reduced degree is formed by a stiffening element applied to the expandable material of said balloon.

7. The stent-catheter arrangement according to any one of claims 1 to 5, characterized in that said section (5) of said balloon (3) which is expandable to a reduced degree is producible during balloon production.
